(19) United States
(12) Patent Application Publication
(10) Pub. No.: US 2024/0420824 A1
(43) Pub. Date: Dec. 19, 2024

(54) 3-Tier Reintegration Formula

(71) Applicant: Lance Collin Allred, Midvale, UT (US)

(72) Inventor: Lance Collin Allred, Midvale, UT (US)

(21) Appl. No.: 18/209,987

(22) Filed: Jun. 14, 2023

(51) Int. Cl. G16H 20/70

(52) U.S. Cl. CPC G16H 20/70

(57) ABSTRACT

The 3-Tier Reintegration Formula is an holistic approach, with its own proprietary intellectual property that will assist the Health Industry in its battle against the opioid epidemic by doing more than just treating the symptom of addiction. It will assist recovery centers by focusing on the root causes of addictions and equipping those battling addiction with career training in professions that move them into alignment with their own core needs and personalities, in three tiers.

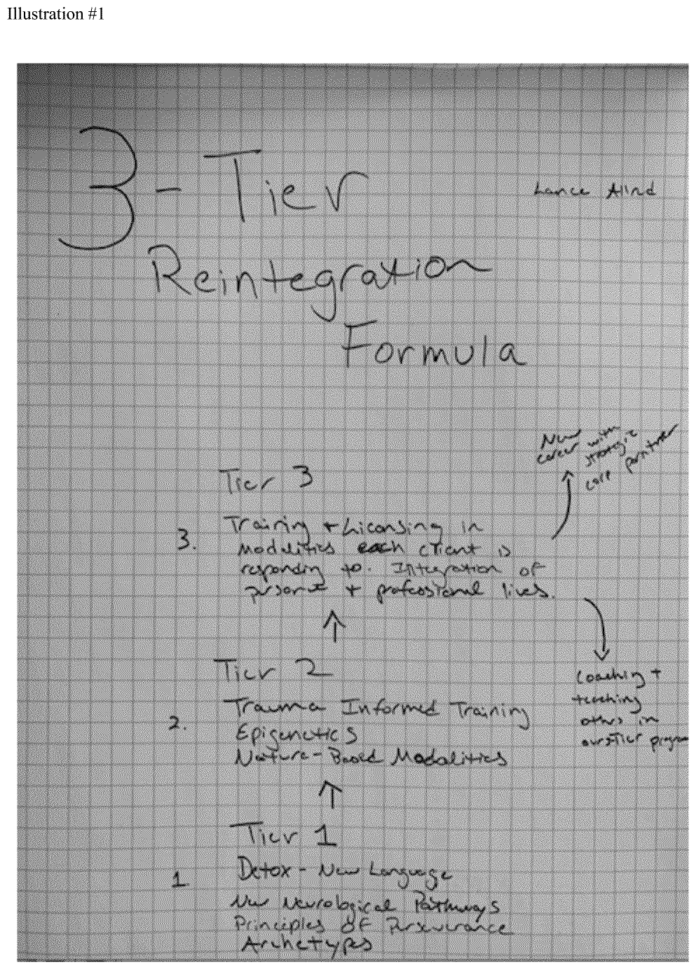

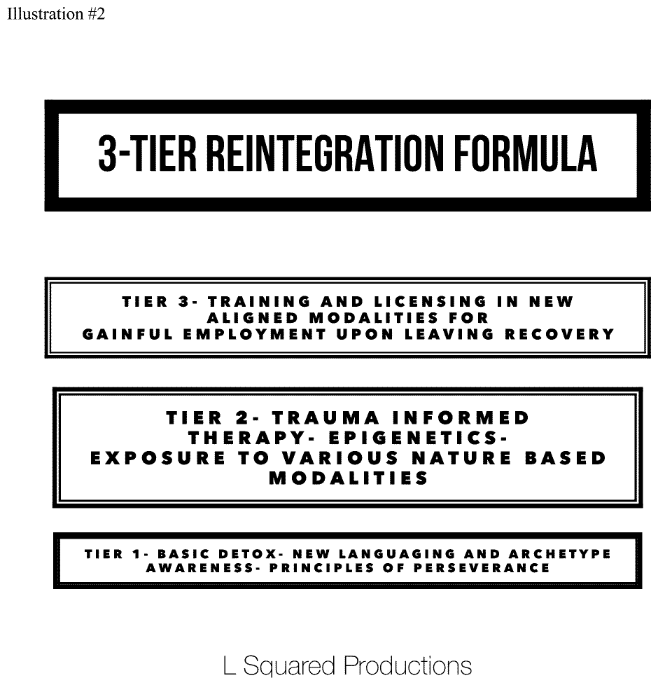

Tier 1 will focus on basic detox and new neuro-pathway development. Tier 2 will focus on trauma informed training and exposure to various nature based modalities. And Tier 3 will focus on training and licensing the clients in the modalities they best respond to, giving them forward momentum as they leave recovery by positioning them with a new career that best amplifies their individual skills. An integrated personal and professional life is essential in helping those struggling with addiction to find balance, ensuring a higher success rate upon leaving recovery

The 3-Tier Reintegration Formula is designed to secure higher success rate in the Health Industry for those battling substance abuse by aligning them with modalities and new professional skills that integrate their personal and work lives into one cohesive and existence.

### Description of Problem(s) that this Invention Solves

“12 Steps” as the most popular post-recovery option is proving to not be enough to battle the Opioid epidemic and showing where the Health Industry is exposed. If there were a one-size-fits-all system we would have found it by now. Substance addiction is at an all time high is evidence of the problem that we are facing and confirmation that we need fresh alternatives and broader thinking-Different people with different personalities and skills need different options to meet their different needs.

### Description on how the Invention is an Improvement Over Existing Technology

While the Health Industry has made strides in medicines that help the symptom of addiction, it is still lacking in the after-care solutions. We need more than just “12 Steps” or Religious-based post-recovery solutions.

Not only will the 3-Tier Reintegration Formula focus on the root of addiction: trauma and shame, it will also focus on offering many modalities to meet different needs of each individual client, with which we will offer to train and license the client with the said modality they are responding best to, upon which they will have job offering with strategic partners or their own entrepreneurship as they leave the 3-Tier Reintegration Recovery Program.

Individual and Business Demographics that would Use this Invention:

Those struggling with substance abuse and PTSD will benefit from this design.

### Description of the Benefits of this Invention to its Users

Integration of one's personal and professional experiences into one fulfilled, cohesive existence is what we are offering. We will focus Emotional intelligence development to help those struggling with addiction be more aware of their own trauma and habits, with forward-momentum and life-changing career opportunities that best align with their own personalities for highest self-fulfillment that will equip them to outgrow old networks and work environments that led to their addiction. Studies confirm that those living in fulfilling work environments are less likely to be triggered back into substance abuse when living an actualized and integrated life that best meets their personality and unique skills.

### BRIEF SUMMARY OF THE INVENTION

3-Tier Reintegration Formula that is built to assist those struggling with substance abuse by focusing on the integration of client's personal and work into one cohesive experience by training and licensing them in the modalities they best respond to in their recovery.

Tier 1-Basic Detox and new languaging and neuro-pathways, some nature-based modalities introduced.

Tier 2-Deprogramming of trauma and shame. More various modalities introduced (0010) Tier 3-Training and licensing for aligned employment in the modality they best respond to as they look to reintegrate back into society as a net-positive member.

### BRIEF DESCRIPTION OF DRAWINGS

The Applicant has attached the following illustrations of the aforementioned invention at the end of this provisional patent application:

Illustration #1 (Page 9) Description: 3-Tier Reintegration Formula Visual Map depicts the structure of how Tier 1 feeds into Tier 2; Tier 2 feeds into Tier 3

Illustration #2 (Page 10) Description: Computer Drawn 3-Tier Reintegration Formula Visual Map depicts the structure of how Tier 1 feeds into Tier 2; Tier 2 feeds into Tier 3

| REFERENCE | |
|---|---|
| NUMERAL | PART NAME |
| 1 | 3-Tier Reintegration Formula Visual Map-Illustrated |
| 2 | 3-Tier Reintegration Formula |
| | Visual Map-Computer Generated |

| Part | |
|---|---|
| Number | PART NAME |
| 1 | Tier 1 |
| 2 | Tier 2 |
| 3 | Tier 3 |

### INVENTION DETAILED DESCRIPTION

Using nature-based settings, most specifically geothermal hot springs settings, the 3-Tier Re-Integration Formula will contribute to the Health Industry by helping those struggling with substance abuse by providing more than just “12 steps,” as the sole post-recovery service. We will be focused on a holistic approach by integrating the personal and professional lives within each client, so they are not living in a compartmentalized, imbalanced or fractured state which studies show only add to addictive tendencies.

### 3-Tier Reintegration Formula Explained:

Tier 1-will provide basic detoxing facilities and foundational groundwork for new thought patterns, language, archetypal awareness and neurological pathway development.

Tier 2-will lay new framework for shame and trauma and help the clients realize their addictive behavior is not only theirs, but rather a family systems and generational issue. From there the client will begin to move with clarity and new skills of insight and self-reflection while being introduced to many new types of therapies and modalities from hypnotherapy to EMDR, to equine therapy, to yoga, to breath work, carpentry, gardening, coding, music and many other modalities and interests.

Tier 3-will be the training and licensing of clients in the modalities they be best respond to, that will set them up with their own entrepreneurial roadmap and/or aligned and gainful employment with one of our strategic corporate partners.

### Description of the Parts of the Invention:

The 3-Tier Reintegration Formula will operate as such:

Tier 1 location-Detox and stabilization. Foundational groundwork of Lance Allred's “Principles of Perseverance.” Survivor archetype awareness and new languaging patterns will be emphasized along with new neurological pathway development. Various modalities will be offered along with the coaching and group/individual therapy by licensed therapists.

Tier 2 location-Trauma focused work, epigenetics, self-discovery of actualized archetypes, skills and talents. Clarity of seeing your addiction is not “yours alone” but the family system's. More nature-based modalities will be integrated.

Tier 3 location-Employment/coaching training and licensing for aligned employment as they begin to re-integrate back into their family and community. The types of skill/job training we will offer and license are listed but not limited to: geothermal engineering, data mining, coding, culinary, hospitality, farming, carpentry, hypnotherapy, breath-work, family constellations, finance/business, coaching, equine therapy, the arts (to be sold in our trading posts), and more. . . . People gaining clarity and empowerment by these said modalities in the self-sustaining geothermal community, will be empowered to teach and be employed by those same resources.

Relationship between the Parts of the Invention:

Tier 1-Filters and feeds those who are serious about their recovery, to Tier 2.

Tier 2-Filters and feeds those who have been successfully de-programmed, and wish to gain new employable skills and licensing to Tier 3.

Tier 3-Actualizes those who have advanced through Tier 1 and Tier 2 with job training and licensing with gainful employment in modalities said clients best responded to, with strategic partnerships with many corporate partners.

### Details of the Invention Operation/Functions:

3-Tier Reintegration Formula will be operated with each addiction recovery location serving specifically as either a “Tier 1,” “Tier 2,” or “Tier 3” location. Licensed and trained therapists and clinicians will supervise each location. Each location will be separate from the other to provide maximum focus for each client's stage of reprogramming and will be community-based with farming, engineering, programming and various other modalities that geothermal locations can offer, to provide those in recovery options that best match their personality and needs.

Tier 1 locations will provide their clients a list of Tier 2 locations they can graduate to for deeper trauma informed training as well as modalities that can further assist their need for daily fulfillment.

Tier 2 locations will provide their clients a list of Tier 3 locations they can graduate to that focus on their preferred modality they are responding best to, where they will be trained and licensed in said modality as a means for a career that is fulfilling and worth pursuing in alignment with their own unique personality.

### Unique Features of Invention:

“12 Steps,” “AA” or Religious-based recovery centers have done their best, admirably so, for the last 100 years. But all systems become outdated and it is essential that we upgrade the standard of how we view addiction recovery, to meet the growing opioid epidemic. We will do this first, by operating at nature-based locations and by not only providing Corporate-level EQ training, but also providing core-aligned licensing and training in many professions that we have strategic partnerships with that will empower our clients with integrated professional and personal core-living upon leaving recovery

### Best Manufacturing Method:

A franchise of nature-based addiction recovery centers that implement the 3-Tier Reintegration Formula, where people can be reconnected with nature and the outside world to tap into their own humanity and develop new skills that best meet their personality strengths to integrate their personal and professional psyches, is essential for biggest impact.

What is claimed is:

1. The 3-Tier Reintegration Formula focuses on more than the symptom addiction, It provides those struggling with addiction an opportunity to integrate various compartmentalized facets of their lives, particularly work and personal, into one cohesive unit, A fulfilling professional life that is in harmony with one's personal life reduces the chances of reversion and substance abuse relapse.
